(19) United States
(12) Patent Application Publication
(10) Pub. No.: US 2012/0107267 A1
(43) Pub. Date: May 3, 2012

(54) INTERLEUKIN-21 VARIANTS HAVING ANTAGONISTIC BINDING TO THE IL-21 RECEPTOR

(75) Inventors: Lishan Kang, Beijing (CN); Kent Bondensgaard, Vaerlose (DK); Siv Annegrethe Hjorth, Virum (DK)

(73) Assignee: Novo Nordisk A/S, Bagsvaerd (DK)

(21) Appl. No.: 13/147,292

(22) PCT Filed: Mar. 10, 2010
(86) PCT No.: PCT/EP10/53026

§ 371 Date: Jan. 16, 2012

Related U.S. Application Data
(60) Provisional application No. 61/210,492, filed on Mar. 19, 2009.
(60) Provisional application No. 61/267,905, filed on Dec. 9, 2009.

(30) Foreign Application Priority Data
Mar. 11, 2009 (EP) 09154853.7
Dec. 2, 2009 (EP) 09177752.4

(51) Int. Cl. C07K 14/54; C12N 15/24; A61K 38/20

(52) U.S. Cl. 424/85.2; 530/351; 536/23.5

(57) ABSTRACT

The invention relates to isolated IL-21 variant peptides having antagonistic binding to the common gamma chain (yC) of the IL-21 receptor, to pharmaceutical compositions comprising said peptides and to the use of said peptides in therapy.

### FIELD OF THE INVENTION

The invention relates to isolated IL-21 variant peptides having antagonistic binding to the common gamma chain (yC) of the IL-21 receptor, to pharmaceutical compositions comprising said peptides and to the use of said peptides in therapy.

### BACKGROUND OF THE INVENTION

Interleukin-21 (IL-21) is a recently identified type 1 cytokine, which is secreted as a 133-amino acid protein by activated CD4⁺ T cells (Parrish-Novak, J. et al., Nature 408, 57-63 (2000)). The IL-21 cytokine has been demonstrated to possess potent stimulatory effects on the proliferation, differentiation and activation of several classes of haematopoietic cells including B-cells, T-cells and NK-cells. The biological effects of IL-21 are mediated via activation of the IL-21 receptor complex, which is composed of an IL-21 private receptor chain (IL-21 Rα) in complex with the common gamma chain (yC), which similarly constitutes an essential component of the signalling receptor complex of the cytokines IL-2, IL-4, IL-7, IL-9, and IL-15. These cytokines thus constitute a subfamily referred to as common gamma chain cytokines, with IL-21 being the most recently added member.

Within the common gamma chain family of cytokines, high resolution structural information has been obtained through X-ray crystallography and NMR spectroscopy for IL-2 and IL-4 (Brandhuber, B. J. et al., Science 238, 1707-1709 (1987), Mott, H. R. et al., Journal of Molecular Biology 247, 979-994 (1995), Powers, R. et al., Science 256, 1673-1677 (1992), Wlodaver, A. et al., Febs Letters 309, 59-64 (1992). It is apparent from these studies that IL-2 and IL-4 along with other type 1 cytokines, including IL-1β, IL-2, IL-4, and GM-CSF, share a common overall topology in their structures in spite of a distant homology in primary sequence. The common structural motif of these proteins consists of a central four-helical bundle, arranged in an up-up-down-down topology, connected by loops which are characterized by a high degree of structural freedom, a considerable difference in loop length, and variation in the number, and positioning, of stabilizing disulfide bridges. In the IL-21 amino acid sequence as shown in SEQ ID No. 1 (a 162 aa long polypeptide), helix A is defined by amino acid residues 41-56; helix B by amino acid residues 69-84; helix C by amino acid residues 92-105; and helix D by amino acid residues 135-148.

Crystal structures have also been reported for IL-2 and IL-4 in complex with the corresponding private chains and, in the case of IL-2, the common gamma chain (Wang, X. Q. et al., Science 310, 1159-1163 (2005), Hage, T. et al., Cell 97, 271-281 (1999)). IL-2 is distinct from both IL-4 and IL-21 by having two private receptor chains, IL-2Rα and IL-2Rβ, where IL-2Rβ is homologous to IL-4Rα and IL-21 Rα. Only minor structural differences are observed between the free and receptor bound forms of IL-2 and IL-4 indicating that only slight structural changes occur for these cytokines upon complex formation. These studies accurately identify the residues of the cytokines involved in receptor binding, and closely mirror earlier results obtained from mutagenesis studies.

IL-4 antagonists have been designed by making variants for which binding to yC has been abolished while preserving binding to the private receptor chain. This was accomplished by a double mutation [R121D, Y124D] in helix D (Tony, H. P. et al., European Journal of Biochemistry 225, 659-665 (1994)). The IL-4 epitope for yC binding have been further explored by biacore analyses with IL-4 variants (Zhang, J. L. et al., European Journal of Biochemistry 269, 1490-1499 (2002). Recently, it has been shown that IL-4 and IL-21 bind to partially overlapping epitopes of yC (Zhang, J. L. et al., Biochemical and Biophysical Research Communications 300, 291-296 (2003)).

By analogy to the IL-4 antagonist ([R121D, Y124D]-IL-4), IL-21 variants with antagonistic properties have been generated by mutation of residues in helix D corresponding to R121 and Y124 in IL-4 (WO 2003/040313). WO 2008/074863 describes a series of IL-21 variants capable of modulating binding to the common gamma chain (yC) of the IL-21 receptor. U.S. Pat. No. 7,186,805 describes a series of IL-21 antagonist molecules, such as [Gln145Asp, Ile148Asp] which corresponds to [Q116D, I119D] as described in SEQ ID No: 2.

Both IL-21 agonism and antagonism have thus been implicated as a potentially useful mechanism for treating diseases and disorders. Generating IL-21 variants having modulated activity can be a useful tool in order to elucidate more about such diseases and disorders and may present potential targets for drug development. As such, there is a continuing need for IL-21 antagonists and a method for designing such.

### SUMMARY OF THE INVENTION

According to a first aspect of the invention there is provided an isolated IL-21 receptor antagonist peptide having a mutation corresponding to Gln-116 in SEQ ID No. 2 characterised in that said peptide additionally comprises a further mutation corresponding to His-120 and/or Leu-123 in SEQ ID No. 2.

The present invention also concerns pharmaceutical compositions comprising such peptides, as well as use of the peptides and/or said preparations in therapy.

The present invention also concerns the use of a peptide according to the present invention or a pharmaceutical composition according to the present invention, wherein the IL-21 peptide is an antagonist of the IL-21 receptor, for use in treating a disease or disorder, wherein said disease or disorder may be treatable by use of an IL-21 antagonist.

The present invention also concerns the use of a peptide according to the present invention, wherein the IL-21 peptide is an antagonist of the IL-21 receptor, for preparation of a pharmaceutical composition for treating a disease or disorder, wherein said disease or disorder may be treatable by use of an IL-21 antagonist.

The present invention also concerns methods for the treatment of a disease or disorder, wherein said disease or disorder may be treatable by use of an IL-21 antagonist, wherein said treatment comprises the administration of an effective amount of an IL-21 peptide according to the present invention, wherein said IL-21 peptide is an antagonist of the IL-21 receptor.

The present invention also concerns a host cell comprising a nucleic acid construct according to the present invention.

The present invention also concerns an antibody that specifically binds a peptide according to the present invention.

### DESCRIPTION OF THE DRAWINGS

FIG. 1: Proliferation of NK92 cells in response to increasing concentrations of IL-21 and mutants [Q116D, L123D], [Q116D, H120D], [Q116D, H120D, L123D] and comparative mutant [Q116D, I119D].

FIG. 2: Competitive inhibition of IL-21 dependent NK92 cell proliferation by mutants [Q116D, L123D], [Q116D, H120D], [Q116D, H120D, L123D] and comparative mutant [Q116D, I119D].

FIG. 3: Competition for binding to the IL-21 Rα between IL-21 and mutants [Q116D, L123D], [Q116D, H120D], [Q116D, H120D, L123D] and comparative mutant [Q116D, I119D] in ALPHA screen binding test.

### DESCRIPTION OF THE SEQUENCES

SEQ ID No. 1: Amino acid sequence for full-length IL-21 (1-162 aa). In this sequence, helix A is defined by amino acid residues 36-55; helix B by amino acid residues 73-81; helix C by amino acid residues 88-102; and helix D by amino acid residues 133-153.

SEQ ID No. 2: Amino acid sequence for h IL-21 (residues 30-162 of SEQ ID No. 1). In this sequence, helix A is defined by amino acid residues 7-26; helix B by amino acid residues 44-52; helix C by amino acid residues 59-73; and helix D by amino acid residues 104-124.

### DESCRIPTION OF THE INVENTION

According to a first aspect of the invention there is provided an isolated IL-21 peptide having a first mutation in an amino acid residue corresponding to Gln-116 in SEQ ID No. 2 characterised in that said peptide additionally comprises a further mutation in one or both amino acid residues corresponding to His-120 and/or Leu-123 in SEQ ID No. 2, or a pharmaceutically acceptable salt, ester, or amide thereof.

The peptides of the invention are IL-21 variants having modulated binding to yC of the IL-21 receptor. In particular, the peptides of the invention have surprisingly found to abolish binding to yC of the IL-21 receptor when compared with previously identified IL-21 antagonist variants. Such abolishment of yC binding is provided by the data presented herein.

The binding of the IL-21 peptide of the invention to the IL-21 receptor may be measured in accordance with Assays 1-3 described herein.

The term peptide includes any suitable peptide and may be used synonymously with the terms polypeptide and protein, unless otherwise stated or contradicted by context; provided that the reader recognize that each type of respective amino acid polymer-containing molecule may be associated with significant differences and thereby form individual embodiments of the present invention (for example, a peptide such as an antibody, which is composed of multiple polypeptide chains, is significantly different from, for example, a single chain antibody, a peptide immunoadhesin, or single chain immunogenic peptide). Therefore, the term peptide herein should generally be understood as referring to any suitable peptide of any suitable size and composition (with respect to the number of amino acids and number of associated chains in a protein molecule). Moreover, peptides in the context of the inventive methods and compositions described herein may comprise non-naturally occurring and/or non-L amino acid residues, unless otherwise stated or contradicted by context.

The term peptide, unless otherwise stated or contradicted by context, (and if discussed as individual embodiments of the term(s) polypeptide and/or protein) also encompasses derivatized peptide molecules. Briefly, in the context of the present invention, a derivative is a peptide in which one or more of the amino acid residues of the peptide have been chemically modified (for instance by alkylation, acylation, ester formation, or amide formation) or associated with one or more non-amino acid organic and/or inorganic atomic or molecular substituents (for instance a polyethylene glycol (PEG) group, a lipophilic substituent (which optionally may be linked to the amino acid sequence of the peptide by a spacer residue or group such as β-alanine, γ-aminobutyric acid (GABA), L/D-glutamic acid, succinic acid, and the like), a fluorophore, biotin, a radionuclide, etc.) and may also or alternatively comprise non-essential, non-naturally occurring, and/or non-L amino acid residues, unless otherwise stated or contradicted by context (however, it should again be recognized that such derivatives may, in and of themselves, be considered independent features of the present invention and inclusion of such molecules within the meaning of peptide is done for the sake of convenience in describing the present invention rather than to imply any sort of equivalence between naked peptides and such derivatives). Non-limiting examples of such amino acid residues include for instance 2-aminoadipic acid, 3-amino-adipic acid, β-alanine, β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-di-aminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methyl-isoleucine, 6-N-methyllysine, N-methylvaline, norvaline, norleucine, ornithine, and statine halogenated amino acids.

IL-21 peptides refer to any peptide that specifically binds to the IL-21 receptor under cellular and/or physiological conditions for an amount of time sufficient to induce, promote, enhance, and/or otherwise modulate a physiological effect associated with the antigen; to allow detection by ELISA, Western blot, or other similarly suitable protein binding technique described herein and/or known in the art and/or to otherwise be detectably bound thereto after a relevant period of time (for instance at least about 15 minutes, at least about 30 minutes, at least about 45 minutes, at least about 1 hour, at least about 2 hours, at least about 4 hours, at least about 6 hours, at least about 12 hours, about 1-24 hours, about 1-36 hours, about 1-48 hours, about 1-72 hours, about one week, or longer). The binding of the IL-21 peptide to the IL-21 receptor may for instance be determined by use of Assays 1-3 as described herein.

In one embodiment, a IL-21 peptide according to the present invention is an analogue of human IL-21.

The term “analogue” as used herein referring to a polypeptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide and/or in-chain. All amino acids for which the optical isomer is not stated are to be understood to mean the L-isomer.

The term “IL-21 analogue” or “analogue of IL-21” or “analogue of human IL-21” as used herein referring to an analogue of IL-21 (or human IL-21), which has the capability of binding to the IL-21 receptor and in particular to the common gamma chain (yC) of the IL-21 receptor.

In one embodiment, an IL-21 peptide of the invention has an amino acid sequence having at least 80% identity to SEQ ID No. 1 or SEQ ID No. 2. In one embodiment, an IL-21 peptide of the invention has an amino acid sequence having at least 85%, such as at least 90%, for instance at least 95%, such as for instance at least 99% identity to SEQ ID No. 1 or SEQ ID No. 2.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity. For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3. times, the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci. USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, an IL-21 peptide of the invention has an amino acid sequence, which sequence is at least 80% similar to SEQ ID No. 1 or SEQ ID No. 2. In one embodiment, an IL-21 peptide of the invention has an amino acid sequence, which sequence is at least 85%, such as at least 90%, for instance at least 95%, such as for instance at least 99% identity to SEQ ID No. 1 or SEQ ID No. 2.

The term “similarity” is a concept related to identity, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides. Conservative modifications a peptide comprising an amino acid sequence of SEQ ID No. 1 or SEQ ID No. 2 (and the corresponding modifications to the encoding nucleic acids) will produce peptides having functional and chemical characteristics similar to those of a peptide comprising an amino acid sequence of SEQ ID No. 1 or SEQ ID No. 2. In contrast, substantial modifications in the functional and/or chemical characteristics of peptides according to the invention as compared to a peptide comprising an amino acid sequence of SEQ ID No. 1 or SEQ ID No. 2 may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a non-native residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl. 643, 55-67 (1998); Sasaki et al., Adv. Biophys. 35, 1-24 (1998), which discuss alanine scanning mutagenesis).

Desired amino acid substitutions (whether conservative or non-conservative) may be determined by those skilled in the art at the time such substitutions are desired. For example, amino acid substitutions can be used to identify important residues of the peptides according to the invention, or to increase or decrease the affinity of the peptides described herein for the receptor in addition to the already described mutations.

Naturally occurring residues may be divided into classes based on common side chain properties:

1) hydrophobic: norleucine, Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157, 105-131 (1982). It is known that certain amino acids may be substituted for other amino acids having a similar hydropathic index or score and still retain a similar biological activity. In making changes based upon the hydropathic index, the substitution of amino acids whose hydropathic indices are within. +−2 is preferred, those that are within +−1 are particularly preferred, and those within +−0.5 are even more particularly preferred.

It is also understood in the art that the substitution of like amino acids may be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine ('3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions”.

Peptides of the present invention may also include non-naturally occurring amino acids.

In one embodiment, the activation of said peptide mediated through the IL-21 receptor is decreased as compared to an IL-21 peptide having the amino acid sequence of SEQ ID No. 2. In one embodiment, the decrease in the binding of said peptide to the IL-21 receptor is at least 2-fold, such as at least 5-fold, for instance at least 10-fold, such as at least 20-fold, for instance at least 50-fold, such as at least 100-fold, for instance at least 500-fold, such as at least 1000-fold as compared to the binding of a IL-21 peptide having the amino acid sequence of SEQ ID No. 2 to the IL-21 receptor.

The decrease in activation through the receptor may be determined by use of for instance the assays described herein as Assays 1-3.

In one embodiment, an IL-21 peptide according to the invention is an antagonist of the IL-21 receptor. In this specification, an antagonist may be a partial agonist or a full antagonist meaning IL-21 peptides that produce either a less efficacious activation or no measurable activation, respectively, when analyzed using Assays 1-3. A less efficacious activation meaning activation corresponding to less than 50% of that achieved at the corresponding dose of the natural agonist, hIL-21. In addition, an antagonist must produce inhibition of the receptor activation mediated by the natural agonist hIL-21 when the former is present at a concentration of 1 nM or less. Thus, in one embodiment, the binding of said peptide to the yC of the IL-21 receptor is decreased compared to an IL-21 peptide having the amino acid sequence of SEQ ID No. 2.

In one embodiment, the introduction of the mutation(s) according to the invention is responsible for or contributory to the antagonistic activity of the IL-21 peptide.

In one embodiment, said further mutation comprises a mutation corresponding to amino acid residue His-120 in SEQ ID No. 2. Thus, the mutant of this embodiment comprises a double mutant having mutations at positions Gln-116 and His-120 (i.e. [Q116, H120]).

In one embodiment, said further mutation comprises a mutation corresponding to amino acid residue Leu-123 in SEQ ID No. 2. Thus, the mutant of this embodiment comprises a double mutant having mutations at positions Gln-116 and Leu-123 (i.e. [Q116, L123]).

In one embodiment, said further mutation comprises two further mutations corresponding to amino acid residues His-120 and Leu-123 in SEQ ID No. 2. Thus, the mutant of this embodiment comprises a triple mutant having mutations at positions Gln-116, His-120 and Leu-123 (i.e. [Q116, H120, L123]).

In one embodiment, an IL-21 peptide according to the invention additionally carries one or more antagonistic mutations in the region corresponding to Helix D of SEQ ID No. 1 as described in for instance Brandt, C et al., Journal of Leukocyte Biology Suppl. S 119, 46-46 (2001). In one embodiment, said additional antagonistic mutations in Helix D is a mutation corresponding to position Ile-119 in SEQ ID No. 2, as described in WO 2003/040313. In one embodiment, Ile-119 has been substituted with an Asp.

In one embodiment, an IL-21 peptide according to the invention additionally carries one or of the mutations as described in CN1513993A. In one embodiment, one or more of said mutations is a mutation in one or more of the amino acid residues corresponding to positions Lys-21 and Arg-83 in SEQ ID No. 2. In one embodiment, Lys-21 has been substituted with a His. In one embodiment, Arg-83 has been substituted with a Gly. In one embodiment, Lys-21 has been substituted with a His and Arg-83 has been substituted with a Gly.

In one embodiment, an IL-21 peptide according to the invention additionally carries one or of the mutations as described in WO 2004/112703.

In one embodiment, an IL-21 peptide according to the invention additionally has a mutation in one or more of the amino acid residues corresponding to Met-7, Arg-11, Ile-14, Asp-18, Glu-36, Asp-37, Thr-40, Glu-100, Glu-109, Ser-113, Lys-117, Ile-119, Ser-125, Arg-126, Thr-127, His-128, Gly-129, Ser-130, Glu-131, Asp-132 and Ser-133 in SEQ ID No. 2.

In one embodiment, an IL-21 peptide according to the invention additionally has a mutation in one or more of the amino acid residues corresponding to Met-7, Arg-11, Ile-14, Asp-18, Glu-100, Glu-109, Ser-113, Lys-117 and Ile-119 in SEQ ID No. 2.

In one embodiment, an IL-21 peptide according to the invention additionally has a mutation in one or more of the amino acid residues corresponding to Met-7, Arg-11, Ile-14, Asp-18, Glu-36, Asp-37, Thr-40, Glu-100, Ser-125, Arg-126, Thr-127, His-128, Gly-129, Ser-130, Glu-131, Asp-132, and Ser-133 in SEQ ID No. 2.

In one embodiment, an IL-21 peptide according to the invention additionally has a mutation in one or more of the amino acid residues corresponding to Arg-11, Glu-36, Asp-37, Thr-40, Glu-100, Ser-113 and Lys-117 in SEQ ID No. 2.

In one embodiment, an IL-21 peptide according to the invention additionally has a mutation in one or more of the amino acid residues corresponding to Ile-14 and Lys-117 in SEQ ID No. 2.

In one embodiment, said peptide additionally comprises a mutation in one or more of the amino acid residues in the region corresponding to Helix A in SEQ ID No. 1. In one embodiment, said peptide comprises a mutation in one or more of the amino acid residues corresponding to positions Met-7, Arg-11, Ile-14 and Asp-18.

In one embodiment, said peptide additionally comprises a mutation in one or more of the amino acid residues in the region corresponding to Helix D in SEQ ID No. 1. In one embodiment, said peptide comprises a mutation in one or more of the amino acid residues corresponding to positions Glu-109, Ser-113, Lys-117 and Ile-119 in SEQ ID No. 2.

In one embodiment, said peptide additionally comprises a mutation in one or more of the amino acid residues in the ten most C-terminal amino acid residues. In one embodiment, said peptide comprises a mutation in one or more of the amino acid residues corresponding to positions Ser-125, Arg-126, Thr-127, His-128, Gly-129, Ser-130, Glu-131, Asp-132, and Ser-133 in SEQ ID No. 2.

In one embodiment, said mutations comprise deletions or substitutions. In one embodiment, said mutations comprise substitutions, such as substitutions with an acidic amino acid residue, such as Asp or Glu, in particular, Asp. Thus, for example, the specific mutants disclosed in the invention are [Q116D, H120D], [Q116D, L123D] and [Q116D, H120D, L123D].

The peptides of the invention may be in the form of a pharmaceutically acceptable salt, amide, or ester. For example, one or more of the free carboxylic acid groups of the peptides of the invention may be in the form of a pharmaceutically acceptable salt, ester, or amide; and/or one or more of the free amino groups may be in the form of a pharmaceutically acceptable salt. In one embodiment, the peptide is in the form of a pharmaceutically acceptable salt. In one embodiment, the peptide is in the form of a pharmaceutically acceptable ester. In one embodiment, the peptide is in the form of a pharmaceutically acceptable amide. Non-limiting examples of salts include salts of NaOH, HCl, TFA (trifluoroacetic acid, CF₃CO₂H), acetic acid, H₂SO₄, and pivalic acid (trimethylacetic acid, CH₃)₃CCO₂H). Non-limiting examples of esters include esters of lower alkyl, straight or branched, having from one to five carbon atoms, for instance from one to three carbon atoms. Non-limiting examples of amides include unsubstituted amide, —CONH₂; mono- or di-substituted amides, N-substituted with lower alkyl, straight or branched, having from one to five carbon atoms, preferably from one to three carbon atoms; as well as the corresponding ammonium salts (such as —CONH₄⁺,C⁻).

The peptides of the present invention may be prepared in different ways. The peptides may be prepared by protein synthetic methods known in the art. Due to the size of the peptides, this may be done more conveniently by synthesising several fragments of the peptides which are then combined to provide the peptides of the present invention. In a particular embodiment, however, the peptides of the present invention are prepared by fermentation of a suitable host comprising a nucleic acid construct encoding the peptides of the present invention. This is well-known by a person skilled in the art.

Peptides according to the present invention may be used in the treatment of different diseases and disorders, where a modulation (such as increasing or a decreasing) IL-21 activity may prove beneficial for the patient. Peptides according to the present invention may be IL-21 antagonists and as such may be useful for treating a variety of diseases and disorders.

The present invention thus provides a peptide according to the present invention for use in therapy.

The present invention also provides the use of a peptide according to the present invention for use in therapy. The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active peptides to prevent the onset of the symptoms or complications. The patient to be treated may be a mammal, in particular a human being, but it may also include animals, such as dogs, cats, cows, sheep and pigs. It is to be understood, that therapeutic and prophylactic (preventive) regimes represent separate aspects of the present invention.

A “therapeutically effective amount” of a peptide as used herein means an amount sufficient to cure, alleviate or partially arrest the clinical manifestations of a given disease and its complications. An amount adequate to accomplish this is defined as “therapeutically effective amount”. Effective amounts for each purpose will depend on the type and severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

Peptides and pharmaceutical compositions according to the present invention, which peptides are IL-21 antagonists may be used in the treatment of a number of diseases and disorders.

Consequently, the present invention also provides the use of a peptide according to the present invention, for use in treating a disease or disorder, wherein said disease or disorder may be treatable by use of an IL-21 antagonist. The present invention also provides the use of a peptide according to the present invention, for the preparation of a pharmaceutical composition for treating a disease or disorder, wherein said disease or disorder may be treatable by use of an IL-21 antagonist. The present invention also provides a method for the treatment of a disease or disorder, wherein said disease or disorder may be treatable by use of an IL-21 antagonist, wherein said treatment comprises the administration of an effective amount of a peptide according to the present invention, to a patient in need thereof.

In one embodiment, such disease or disorder is an autoimmune and/or inflammatory disease. Examples of such autoimmune and/or inflammatory diseases are Systemic Lupus Erythematosus (SLE), Rheumatoid Arthritis (RA) and inflammatory bowel disease (IBD) (including ulcerative colitis (UC) and Crohn's disease (CD)), multiple sclerosis (MS), scleroderma and type 1 diabetes (T1D), and other diseases and disorders, such as PV (pemphigus vulgaris), psoriasis, atopic dermatitis, celiac disease, kol, hashimoto's thyroiditis, graves' disease (thyroid), Sjogren's syndrome, guillain-barre syndrome, goodpasture's syndrome, additon's disease, Wegener's granulomatosis, primary biliary sclerosis, sclerosing cholangitis, autoimmune hepatitis, polymyalgia rheumatica, paynaud's phenomenon, temporal arteritis, giant cell arteritis, autoimmune hemolytic anemia, pernicious anemia, polyarteritis nodosa, behcet's disease, primary bilary cirrhosis, uveitis, myocarditis, rheumatic fever, ankylosing spondylitis, glomerulenephritis, sarcoidosis, dermatomyositis, myasthenia gravis, polymyositis, alopecia greata, and vitilgo. Other examples can be found in PCT application WO 01/46420, which is directed at the use of IL-17 for treatment of autoimmune and/or inflammatory diseases and wherein several examples of such diseases are given.

In one embodiment, such disease or disorder is SLE, RA or IBD.

In one embodiment, such disease or disorder is MS.

The IL-21 peptides of the present invention may be administered in combination with other medicaments as is known in the art.

With regard to the antagonistic IL-21 peptides of the invention and the treatment of autoimmune diseases, such combination therapy may include administration of an IL-21 peptide of the present invention together with a medicament, which together with the IL-21 peptide comprise an effective amount for preventing or treating such autoimmune diseases. Where said autoimmune disease is Type 1 diabetes, the combination therapy may encompass one or more of an agent that promotes the growth of pancreatic beta-cells or enhances beta-cell transplantation, such as beta cell growth or survival factors or immunomodulatory antibodies. Where said autoimmune disease is rheumatoid arthritis, said combination therapy may encompass one or more of methotrexate, an anti-TNF-α antibody, aTNF-α receptor-Ig fusion protein, an anti-IL-15 antibody, a non-steroidal anti-inflammatory drug (NSAID), or a disease-modifying anti-rheumatic drug (DMARD). For example, the additional agent may be a biological agent such as an anti-TNF agent (e.g., Enbrel®, infliximab (Remicade®) and adalimumab (Humira®) or rituximab (Rituxan®). Where said autoimmune disease is hematopoietic transplant rejection, hematopoietic growth factor(s) (such as erythropoietin, G-CSF, GM-CSF, IL-3, IL-11, thrombopoietin, etc.) or antimicrobial(s) (such as antibiotic, antiviral, antifungal drugs) may be administered. Where said autoimmune disease is psoriasis, the additional agent may be one or more of tar and derivatives thereof, phototherapy, corticosteroids, Cyclosporine A, vitamin D analogs, methotrexate, p38 mitogen-activated protein kinase (MAPK) inhibitors, as well as biologic agents such as anti-TNF-α agents and Rituxan®. Where said autoimmune disease is an inflammatory bowel disease (IBD) such as, for example, Crohn's Disease or ulcerative colitis, the additional agent may be one or more of aminosalicylates, corticosteroids, immunomodulators, antibiotics, or biologic agents such as Remicade® and Humira®.

The combination treatment may be carried out in any way as deemed necessary or convenient by the person skilled in the art and for the purpose of this specification, no limitations with regard to the order, amount, repetition or relative amount of the compounds to be used in combination is contemplated.

Accordingly, the IL-21 peptides according to the present invention for use in therapy may be formulated into pharmaceutical compositions. The present invention is also related to pharmaceutical compositions comprising peptides according to the present invention. Pharmaceutical compositions according to the present invention may be administered alone or in combination with pharmaceutically acceptable carriers or excipients, in either single or multiple doses. The formulation of the combination may be as one dose unit combining the compounds, or they may be formulated as separate doses. The pharmaceutical compositions comprising IL-21 variants according to the present invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995. The compositions may appear in conventional forms, for example capsules, tablets, aerosols, solutions or suspensions.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen. The route of administration may be any route, which effectively transports the active compound to the appropriate or desired site of action.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, such as from about 0.01 to about 50 mg/kg body weight per day, for example from about 0.05 to about 10 mg/kg body weight per day administered in one or more dosages such as 1 to 3 dosages. The exact dosage will depend upon the nature of the IL-21 polypeptide chosen, the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, for example from about 0.1 to about 500 mg, such as from about 0.5 mg to about 200 mg.

For parenteral routes such as intravenous, intrathecal, intramuscular and similar administration, typically doses are in the order of about half the dose employed for oral administration.

Salts of IL-21 variants according to the present invention are especially relevant when the peptide is in solid or crystalline form. For parenteral administration, solutions of the IL-21 variants according to the present invention in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining a IL-21 variant according to the present invention and the pharmaceutically acceptable carriers are then readily administered in a variety of dosage forms suitable for the disclosed routes of administration. The formulations may conveniently be presented in unit dosage form by methods known in the art of pharmacy.

For nasal administration, the preparation may contain a IL-21 variant according to the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Formulations of IL-21 variants according to the present invention, optionally together with the combination agent suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the IL-21 variants according to the present invention, optionally together with the combination agent in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid. Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions of IL-21 variants according to the present invention, optionally together with the combination agent may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, preservatives and flavouring and colouring agents. The pharmaceutical compositions may be in the form of a sterile injectible aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example. For topical use, creams, ointments, jellies, solutions of suspensions, etc., containing the compounds of the invention are contemplated. For the purpose of this application, topical applications shall include mouth washes and gargles.

The IL-21 variants according to the present invention, optionally together with the combination agent may also be administered in the form of liposome delivery systems, such as small unilamellar vesicles, large unilamellar vesicles, and multilamellar vesicles. Liposomes may be formed from a variety of phospholipids, such as cholesterol, stearyl-amine, or phosphatidylcholines.

In addition, some of the IL-21 variants according to the present invention may form solvates with water or common organic solvents. Such solvates are also encompassed within the scope of the invention.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

The IL-21 variants according to the present invention, optionally together with the combination agent may be administered to a mammal, especially a human, in need of such treatment. Such mammals include also animals, both domestic animals, e.g. household pets, and non-domestic animals such as wildlife.

Pharmaceutical compositions containing an IL-21 variant according to the present invention may be administered one or more times per day or week, for instance at mealtimes. An effective amount of such a pharmaceutical composition is the amount that provides a clinically significant effect. Such amounts will depend, in part, on the particular condition to be treated, age, weight, and general health of the patient, and other factors evident to those skilled in the art.

The present invention also provides an isolated nucleic acid construct encoding a peptide according to the present invention.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a peptide of interest. The construct may optionally contain other nucleic acid segments. A nucleic acid construct of the invention may suitably be of genomic or cDNA origin, for instance obtained by preparing a genomic or cDNA library and screening for DNA sequences coding for all or part of the peptide by hybridization using synthetic oligonucleotide probes in accordance with standard techniques (cf. J. Sambrook et al, 1989, Molecular Cloning, A Laboratory Manual, 2d edition, Cold Spring Harbor, N.Y.) and by introducing the relevant mutations as it is known in the art.

A nucleic acid construct of the invention may also be prepared synthetically by established standard methods, e.g. the phosphoamidite method described by Beaucage and Caruthers, Tetrahedron Letters 22, 1859-1869 (1981), or the method described by Matthes et al., EMBO Journal 3, 801-805 (1984). According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239, 487-491 (1988).

In one embodiment, the nucleic acid construct of the invention is a DNA construct which term will be used exclusively in the following for convenience. The statements in the following may also read on other nucleic acid constructs of the invention with appropriate adaptions as it will be clear for a person skilled in the art.

In one embodiment, the present invention relates to a recombinant vector comprising a DNA, or nucleic acid, construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The vector may be an expression vector in which the DNA sequence encoding the peptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the peptide.

The promoter may be any DNA sequence which shows transcriptional activity in the host cell of choice and may be derived from genes encoding proteins either homologous or heterologous to the host cell.

Examples of suitable promoters for use in yeast host cells include promoters from yeast glycolytic genes (Hitzeman et al., J. Biol. Chem. 255, 12073-12080 (1980); Alber and Kawasaki, J. Mol. Appl. Gen. 1, 419-434 (1982)) or alcohol dehydrogenase genes (Young et al., in Genetic Engineering of Microorganisms for Chemicals (Hollaender et al, eds.), Plenum Press, New York, 1982), or the TPM (U.S. Pat. No. 4,599,311) or ADH2-4-c (Russell et al., Nature 304, 652-654 (1983)) promoters.

Examples of suitable promoters for use in filamentous fungus host cells are, for instance, the ADH3 promoter (McKnight et al., The EMBO J. 4, 2093-2099 (1985)) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. In one embodiment, the promoter of a vector according to the invention is selected from the TAKA-amylase or the gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the peptide of the invention may also, if necessary, be operably connected to a suitable terminator, such as the human growth hormone terminator (Palmiter et al., op. cit.) or (for fungal hosts) the TPM (Alber and Kawasaki, op. cit.) or ADH3 (McKnight et al., op. cit.) terminators. The vector may further comprise elements such as polyadenylation signals (e.g. from SV40 or the adenovirus 5 E1b region), transcriptional enhancer sequences (e.g. the SV40 enhancer) and translational enhancer sequences (e.g. the ones encoding adenovirus VA RNAs). The recombinant vector of the invention may further comprise a DNA sequence enabling the vector to replicate in the host cell in question.

When the host cell is a yeast cell, suitable sequences enabling the vector to replicate are the yeast plasmid 2p replication genes REP 1-3 and origin of replication.

When the host cell is a bacterial cell, sequences enabling the vector to replicate are DNA polymerase Ill complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 125-130 (1985)), or one which confers resistance to a drug, e.g. ampicillin, kanamycin, tetracyclin, chloramphenicol, neomycin, hygromycin or methotrexate. For filamentous fungi, selectable markers include amdS, pyrG, arqB, niaD and sC.

To direct a peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

For secretion from yeast cells, the secretory signal sequence may encode any signal peptide which ensures efficient direction of the expressed peptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 643-646 (1981)), a modified carboxypeptidase signal peptide (cf. L. A. Valls et al., Cell 48, 887-897 (1987)), the yeast BAR1 signal peptide (cf. WO 87/02670), or the yeast aspartic protease 3 (YAP3) signal peptide (cf. M. Egel-Mitani et al., Yeast 6, 127-137 (1990)).

For efficient secretion in yeast, a sequence encoding a leader peptide may also be inserted downstream of the signal sequence and upstream of the DNA sequence encoding the peptide. The function of the leader peptide is to allow the expressed peptide to be directed from the endoplasmic reticulum to the Golgi apparatus and further to a secretory vesicle for secretion into the culture medium (i.e. exportation of the peptide across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide may be derived from a gene encoding A. oryzae TAKA amylase, A. niger neutral α-amylase, A. niger acid-stable amylase, or A. niger glucoamylase.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., op.cit.).

The host cell into which the DNA construct or the recombinant vector of the invention is introduced may be any cell which is capable of producing the present peptide and includes bacteria, yeast, fungi and higher eukaryotic cells. The present invention also related to a host cell comprising a nucleic acid construct according to the present invention, or a vector according to the present invention.

Examples of bacterial host cells which, on cultivation, are capable of producing the peptide of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gram negative bacteria such as Escherichia coli. The transformation of the bacteria may be effected by protoplast transformation or by using competent cells in a manner known per se (cf. Sambrook et al., supra). Other suitable hosts include S. mobaraense, S. lividans, and C. glutamicum (Appl. Microbiol. Biotechnol. 64, 447-454 (2004)).

When expressing the peptide in bacteria such as E. coli, the peptide may be retained in the cytoplasm, typically as insoluble granules (known as inclusion bodies), or may be directed to the periplasmic space by a bacterial secretion sequence. In the former case, the cells are lysed and the granules are recovered and denatured after which the peptide is refolded by diluting the denaturing agent. In the latter case, the peptide may be recovered from the periplasmic space by disrupting the cells, e.g. by sonication or osmotic shock, to release the contents of the periplasmic space and recovering the peptide. Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. An example of a vector for use in yeast is the POT1 vector disclosed in U.S. Pat. No. 4,931,373. The DNA sequence encoding the peptide of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 3459-3465 (1986); U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al. Gene 78, 147-156 (1989).

When a filamentous fungus is used as the host cell, it may be transformed with the DNA construct of the invention, conveniently by integrating the DNA construct in the host chromosome to obtain a recombinant host cell. This will make it more likely that the DNA sequence will be stably maintained in the cell. Integration of the DNA constructs into the host chromosome may be performed according to conventional methods, e.g. by homologous or heterologous recombination.

The transformed or transfected host cell described above is then cultured in a suitable nutrient medium under conditions permitting the expression of the present peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of peptide in question.

Peptides of the present invention may be used to raise antibodies that specifically bind to the peptides of the present invention. In the present context, “antibodies” include monoclonal and polyclonal antibodies, and antigen-binding fragments thereof, such as F(ab′)₂ and Fab fragments, including genetically engineered antibodies and humanized antibodies. Antibodies are said to be specific if they bind to a peptide of the present invention with a Kₐ greater than or equal to 10⁷ M⁻¹. Methods for preparing antibodies are disclosed in e.g. Hurrell J. G. R. (Ed.) Monoclonal Hybridoma Antibodies: Techniques and Applications, CRC Press, Boca Raton, Fla., 1982 and Sambrok, Molecular Cloning: A Laboratory Manual, Cold Spring Harbour, New York, 1989.

In one embodiment, the invention relates to a specific antibody against a peptide of the present invention. In one embodiment, said antibody does not bind to hIL-21 or Met-hIL-21 or to any of the polypeptides described in International Application WO 2004/112703 or any of the other prior art IL-21 peptides as described herein.

The following is a non-limiting list of embodiments of the present invention.

Embodiment 1: An isolated IL-21 peptide having a first mutation in an amino acid residue corresponding to Gln-116 in SEQ ID No. 2 characterised in that said peptide additionally comprises a further mutation in one or both amino acid residues corresponding to His-120 and/or Leu-123 in SEQ ID No. 2.

Embodiment 2: An isolated peptide according to embodiment 1, wherein said further mutation comprises a mutation in His-120 in SEQ ID No. 2.

Embodiment 3: An isolated peptide according to embodiment 2, wherein said mutation is a deletion mutation or substitution mutation.

Embodiment 4: An isolated peptide according to embodiment 2 or embodiment 3, wherein said mutation is a substitution mutation.

Embodiment 5: An isolated peptide according to any of embodiments 2 to 4, wherein said mutation is a substitution with an acidic amino acid residue.

Embodiment 6: An isolated peptide according to any of embodiments 2 to 5, wherein said mutation is a substitution with Asp or Glu.

Embodiment 7: An isolated peptide according to any of embodiments 2 to 6, wherein said mutation is a substitution with Asp.

Embodiment 8: An isolated peptide according to embodiment 1, wherein said further mutation comprises a mutation in Leu-123 in SEQ ID No. 2.

Embodiment 9: An isolated peptide according to embodiment 8, wherein said mutation is a deletion mutation or substitution mutation.

Embodiment 10: An isolated peptide according to embodiment 8 or embodiment 9, wherein said mutation is a substitution mutation.

Embodiment 11: An isolated peptide according to any of embodiments 8 to 10, wherein said mutation is a substitution with an acidic amino acid residue.

Embodiment 12: An isolated peptide according to any of embodiments 8 to 11, wherein said mutation is a substitution with Asp or Glu.

Embodiment 13: An isolated peptide according to any of embodiments 8 to 12, wherein said mutation is a substitution with Asp.

Embodiment 14: An isolated peptide according to embodiment 1, wherein said further mutations comprises a mutation in His-120 and a mutation in Leu-123 in SEQ ID No. 2.

Embodiment 15: An isolated peptide according to embodiment 14, wherein said further mutations are a deletion mutation and/or a substitution mutation.

Embodiment 16: An isolated peptide according to embodiment 14 or 15, wherein said further mutations are substitution mutations.

Embodiment 17: An isolated peptide according to any of embodiments 14 to 16, wherein said further mutations are substitution mutations with an acidic amino acid residue.

Embodiment 18: An isolated peptide according to any of embodiments 14 to 17, wherein said further mutations are substitution mutations with Asp or Glu.

Embodiment 19: An isolated peptide according to any of embodiments 14 to 18, wherein said further mutations are substitution mutations with Asp.

Embodiment 20: An isolated peptide according to any of embodiments 1 to 19, which additionally comprises a further mutation in one or more amino acid residues corresponding to: Met-7, Arg-11, Ile-14, Asp-18, Glu-36, Asp-37, Thr-40, Glu-100, Glu-109, Ser-113, Lys-117, Ile-119, Ser-125, Arg-126, Thr-127, His-128, Gly-129, Ser-130, Glu-131, Asp-132 and Ser-133 in SEQ ID No. 2.

Embodiment 21: An isolated peptide according to any of embodiments 1 to 20, wherein said peptide is an antagonist of the IL-21 receptor.

Embodiment 22: An isolated peptide according to embodiment 21, wherein the binding of said peptide to the yC of the IL-21 receptor is decreased compared to an IL-21 peptide having the amino acid sequence of SEQ ID No. 2.

Embodiment 23: An isolated IL-21 peptide according to any of embodiments 1 to 22 for use in therapy.

Embodiment 24: A pharmaceutical composition comprising a peptide according to any of embodiments 1 to 22.

Embodiment 25: Use of a peptide according to any of embodiments 1 to 22 or a pharmaceutical composition according to embodiment 24 for use in therapy.

Embodiment 26: Use of a peptide according to any of embodiments 1 to 22, wherein the IL-21 peptide is an antagonist of the IL-21 receptor, for the preparation of a pharmaceutical composition for use in treating a disease or disorder, wherein said disease or disorder may be treatable by use of an IL-21 antagonist.

Embodiment 27: Use of a peptide according to any of embodiments 1 to 22 or a pharmaceutical composition according to embodiment 24, wherein the IL-21 peptide is an antagonist of the IL-21 receptor, for use in treating a disease or disorder, wherein said disease or disorder may be treatable by use of an IL-21 antagonist.

Embodiment 28: Use according to embodiment 26 or embodiment 27, wherein said disease or disorder is an autoimmune and/or inflammatory disease.

Embodiment 29: Use according to embodiment 28, wherein said disease or disorder is systemic lupus erythematosus, rheumatoid arthritis or inflammatory bowel disease.

Embodiment 30: A method of treating a disease or disorder, wherein said disease or disorder may be treatable by use of an IL-21 antagonist, comprising administering to a subjecta peptide according to any of embodiments 1 to 22 or a pharmaceutical composition according to embodiment 24 in an amount effective to treat or prevent the disease.

Embodiment 31: A method according to embodiment 30, wherein the disease or disorder is an autoimmune and/or inflammatory disease.

Embodiment 32: A method according to embodiment 30, wherein the disease or disorder is systemic lupus erythematosus, rheumatoid arthritis or inflammatory bowel disease.

Embodiment 33: An isolated nucleic acid construct encoding a peptide according to any of embodiments 1 to 22.

Embodiment 34: An antibody, which specifically binds a peptide according to any of embodiments 1 to 22.

### EXAMPLES

### Methodology

A full-length cDNA of hIL-21 including a C-terminal HA epitope (YPYDVPDYA), the latter included for the purpose of determining concentration, was inserted into the pcDNA3.1(+) vector to construct a eukaryotic expression plasmid. Site-directed mutagenesis was performed on the pcDNA3.1(+)/hIL-21HA plasmid using a QuickChange® mutagenesis kit (Stratagene) to create hlL-21 double or triple mutants. DNA sequencing was subsequently used to confirm the integrity of the mutants.

Plasmid DNA encoding the respective proteins was transfected with Lipofectamine™ 2000 (Invitrogen) into FreeStyle HEK293 cells. For protein production, cells were grown in serum free FreeStyle 293 medium containing 4 mM glutamine, 1% PLURONIC® F68 and Penicillin Streptomycin antibiotics at 1×10⁶ cells per ml and incubated for 3 days at 37° C., 8% CO₂ with constant shaking. Supernatants were pooled and concentrated by ultrafiltration.

The concentration of the IL-21-HA fusion proteins was determined by an AlphaScreen® HA (Hemagglutinin) Detection Kit (PerkinElmer Life Sciences) and performed in triplicate in 96-well white opaque half-area plates (PerkinElmer) as follows. First, 15 μl of biotinylated-HA (30 nM final concentration) was incubated with decreasing concentrations of hIL-21HA variants, prepared by serial dilution in binding buffer. After 10 minutes, 10 μl anti-HA acceptor beads (1:100 dilution) were added to each well and incubated for 60 min at room temperature. Then, 10 μl streptavidin-coated donor beads (1:100 dilution) were added to each well and incubated for 60 min at room temperature. All addition and incubations were made in subdued lighting conditions due to photosensitivity of the beads. The assay was measured on an EnVision™ microplate analyzer.

### Example 1

### Binding studies comprising [Q116D, H120D], [Q116D, L123D] and [Q116D, H120D, L123D]

### (a) NK92 Proliferation Assay

NK92 is a human NK cell line dependent on IL-2 or IL-21. In the absence of IL-2, the NK92 cells will, when exposed to IL-21, survive and proliferate, while cells cease proliferation and die within a few days without the stimulation of IL-21. The proliferation rate of NK92 is closely correlated to the activity unit of IL-21. The higher activity of IL-21 that the cells are exposed to, the greater the rate of cellular proliferation. Proliferation of NK92 cells can therefore be used as an assay for biological activity of IL-21 and IL-21 variants.

The NK92 cells were obtained from the American Type Tissue Collection and were cultured in MyeloCult™ (MyeloCult™ 5100, StemCell Inc, cat. nr. 05150) supplemented with 150 units/ml of IL-2 (Chemicon Cat.no. IL002), and penicillin-streptomycin; grown at 37° C. and 5% CO₂; and passaged every 48 h. For IL-2 starvation, NK92 cells were plated in the absence of IL-2 for 12-16 h prior to hIL-21 or variant stimulation. Next 10⁵ cells/80 μl/well were seeded in 96-well plates, followed by adding 20 μl of hIL-21 variant at different concentrations. All of the samples were triplicated. After 3 days in culture, each well was added 20 μl Alama-Blue™ (Serotec, U.K.). Six hours later, fluorescence was measured at excitation wavelength of 530 nm and fluorescence wavelength of 590 nm using multilabel counter (Wallac-Berthold, Japan). Data analysis was performed using GraphPad Prism.

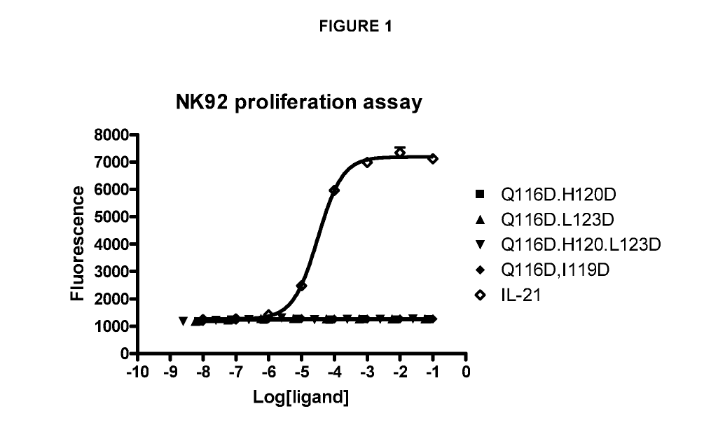

The results of the NK92 proliferation assay are shown in FIG. 1 wherein it can be seen that both double mutants [Q116D, H120D] and [Q116D, L123D] and the triple mutant [Q116D, H120D, L123D] each failed to induce the proliferation of NK92 cells.

### (b) Competitive NK92 Proliferation Assay

After 12-16 h IL-2 starvation, NK92 cells were cultured in MyeloCult™ containing wild type IL-21 with the concentration of EC50. Next 10⁵ cells/80 μl/well were seeded in 96-well plates, followed by adding 20 μl of hIL-21 variant at different concentrations. All of the samples were treated and tested as described in section (a) hereinbefore.

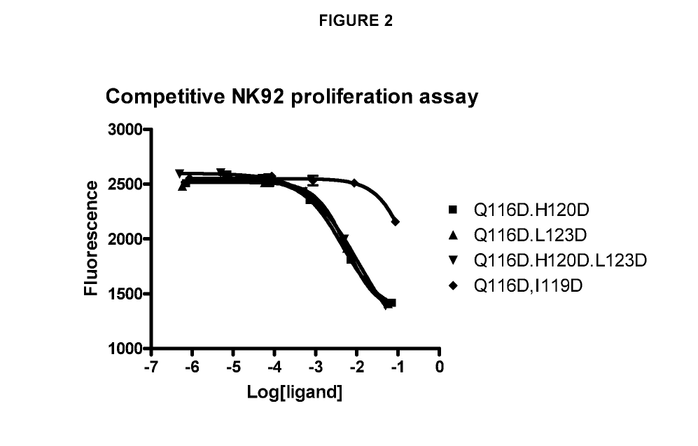

The results of the competitive NK92 proliferation assay are shown in FIG. 2 wherein it can be seen that both double mutants [Q116D, H120D] and [Q116D, L123D] and the triple mutant [Q116D, H120D, L123D] each inhibited the induction of wild type hIL-21 to NK92 cells in a dose-dependent manner.

### (c) IL-21Rα Binding Assay

The affinity of the IL-21HA mutants towards the hIL-21Rα extracellular domain was determined using an ALPHAScreen assay and performed in triplicate in 96-well white opaque half-area plates (PerkinElmer) as follows. First, 15 μl of biotinylated hIL-21 HA (30 nM final concentration) was incubated with decreasing concentrations of hIL-21 HA mutants, prepared by serial dilution in binding buffer. After 10 minutes, 15 μl His6-tagged receptor EC domain (final concentration 30 nM) was added to each well and incubated for 30 min at room temperature. Then 10 μl Ni²⁺ chelating acceptor beads (1:100 dilution) were added to each well and incubated for 60 min at room temperature. Finally, 10 μl streptavidin-coated donor beads (1:100 dilution) were added to each well and incubated for 60 min at room temperature. All additions and incubations were done under subdued lighting conditions due to photosensitivity of the beads. The assay was measured on an EnVision™ microplate analyzer.

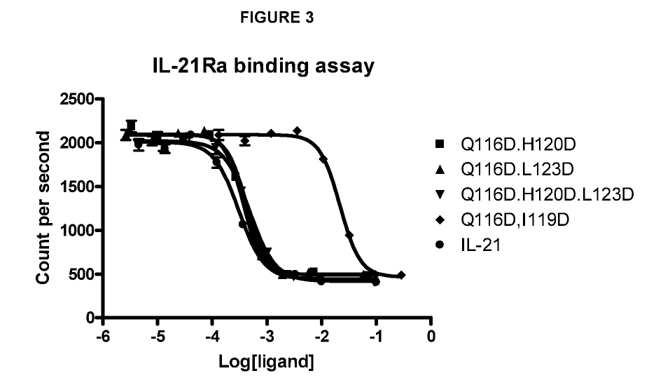

The results of the IL-21Rα binding assay are shown in FIG. 3 wherein it can be seen that both double mutants [Q116D, H120D] and [Q116D, L123D] and the triple mutant [Q116D, H120D, L123D] were found to bind to hIL-21Rα equally well as wild-type hIL-21.

The results of the binding studies conducted in Example 1 demonstrate that both double mutants [Q116D, H120D] and [Q116D, L123D] and the triple mutant [Q116D, H120D, L123D] each acted as full antagonists of the hIL-21 wild-type cytokine.

### Example 2

### Comparative Binding Study Comprising [Q116D, I119D]

[Q116D, I119D] was previously reported in U.S. Pat. No. 7,186,805. This experiment was intended to provide a comparison with the mutants of the present invention which all share the Q116D mutation. In the NK92 proliferation assay neither antagonist provided any measurable activity (see FIG. 1). However, in the inhibitory competition assay, the effect of [Q116D, I119D] was in the order of 100 times lower than that of the two double mutants and the triple mutant of the invention ([Q116D, H120D], [Q116D, L123D] and [Q116D, H120D, L123D]) (see FIG. 2). According to this analysis of receptor binding, the I119D mutant has a dramatically reduced binding affinity to the yC receptor chain, however, in contrast to the H120D and L123D mutations included in the mutants of the invention, the mutation I119D also results in a significantly reduced affinity towards the hIL-21Rα chain, which in turn decreases the affinity of [Q116D, I119D] towards hIL-21Rα (see FIG. 3). The selective elimination of yC binding is therefore a unique characteristic of the mutants of the invention.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

1. An isolated IL-21 peptide having a first mutation in an amino acid residue corresponding to Gln-116 in SEQ ID No. 2 and at least one further mutation in an amino acid residue corresponding in SEQ ID NO: 2 to His-120, and/or Leu-123, or both.

2. The isolated peptide according to claim 1, wherein said further mutation comprises a mutation in His-120 in SEQ ID No. 2.

3. The isolated peptide according to claim 2, wherein said further mutation is a Asp for His or a Glu for His substitution.

4. The isolated peptide according to claim 1, wherein said further mutation comprises a mutation in Leu-123 in SEQ ID No. 2.

5. The isolated peptide according to claim 4, wherein said further mutation is a Asp for Leu or Glu for Leu substitution mutation.

6. The isolated peptide according to claim 1, wherein said further mutation comprises a mutation in His-120 and a mutation in Leu-123 in SEQ ID No. 2.

7. The isolated peptide according to claim 6, wherein said further mutation substitutes Asp or Glu for His-120 or Leu-123.

8. The isolated peptide according to claim 6, wherein said further mutation substitutes Asp for His-120 or Leu-123.

9. The isolated peptide according to claim 1, wherein said peptide is an antagonist of the IL-21 receptor.

10. (canceled)

11. A pharmaceutical composition comprising a peptide according to claim 1.

12. (canceled)

13. A method for treating a disease or disorder treatable by an IL-21 antagonist comprising administering the peptide of claim 1 to a patient in need thereof, wherein the IL-21 peptide is an antagonist of the IL-21 receptor.

14. A method for treating a disease or disorder treatable by an IL-21 antagonist comprising administering the pharmaceutical composition according to claim 11 to a patient in need thereof, wherein the IL-21 peptide is an antagonist of the IL-21 receptor.

15. An isolated nucleic acid construct encoding a peptide according to claim 1.


SEQUENCE LISTING (2 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 133 aa, Homo sapiens [sequence omitted]